(19) United States
(12) Patent Application Publication
(10) Pub. No.: US 2008/0077156 A1
(43) Pub. Date: Mar. 27, 2008

(54) SURGICAL DISTRACTOR AND DELIVERY INSTRUMENT

(76) Inventor: Erik Emstad, St. Paul, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/861,010

(22) Filed: Sep. 25, 2007

Related U.S. Application Data
(60) Provisional application No. 60/846,944, filed on Sep. 25, 2006.

(51) Int. Cl. A61B 17/58

(52) U.S. Cl. 606/105; 606/90

(57) ABSTRACT

A surgical distractor for distracting a joint space to facilitate passage of a surgical tool. The distractor includes a first arm, a second arm, a coupling device, and a limit device. Each arm includes a handle portion and a flange. The coupling device pivotably couples the first and second arms such that the flanges combine to define a passage. A size of the passage increases when transitioning the distractor from a first state of expansion to a second state of expansion. The limit device is associated with the arms for selectively preventing transitioning of the distractor from the second state of expansion to the first state of expansion. In some embodiments, the flanges, and thus the passage, is laterally and/or angularly offset from the handle portions.

### CROSS-REFERENCE TO RELATED APPLICATIONS

The subject matter of this application is related to the subject matter of U.S. Provisional Application Ser. No. 60/846,944, filed Sep. 25, 2006 and entitled “Prosthesis Insertion Instrument;” priority to which is claimed under 35 U.S.C. §119(e) and an entirety of which is incorporated herein by reference.

### BACKGROUND

The present disclosure relates to surgical devices and methods associated with facilitating delivery of surgical tools to a bodily joint, for example implanting a spinal prosthetic into a spinal disc space.

Many surgical procedures entail accessing an enclosed anatomical structure, such as a bodily joint, and delivering a surgical tool to the enclosed area. For example, prosthetic implants are commonly used for repairing a plethora of different anatomical structures and joints. Implanting a spinal prosthesis is representative of the difficulties associated with many of these procedures. As a point of reference, the vertebrate spine is the axis of the skeleton on which all of the body parts “hang.” In humans, the normal spine has seven cervical, twelve thoracic and five lumbar segments. The lumbar spine sits upon the sacrum, which then attaches to the pelvis, and in turn, is supported by the hip and leg bones. The bony vertebral bodies of the spine are separated by intervertebral discs, which act as joints but allow known degrees of flexion, extension, lateral bending, and axial rotation.

The typical vertebra has a thick anterior bone mass called the vertebral body, with a neural (vertebral) arch that arises from the posterior surface of the vertebral body. The centra of adjacent vertebrae are supported by intervertebral discs. Each neural arch combines with the posterior surface of the vertebral body and encloses a vertebral foramen. The vertebral foramina of adjacent vertebrae are aligned to form a vertebral canal, through which the spinal sac, cord, and nerve rootlets pass. The portion of the neural arch which extends posteriorly and acts to protect the spinal cord's posterior side is known as the lamina. The spineous process projects from the posterior region of the neural arch.

The intervertebral disc primarily serves as a mechanical cushion permitting controlled motion between vertebral segments of the axial skeleton. The normal disc is a unique, mixed structure, comprised of three component tissues: the nucleus pulpous (“nucleus”), the annulus fibrosus (“annulus”) and two vertebral end plates. The two vertebral end plates are composed of thin cartilage overlying a thin layer of hard, cortical bone which attaches to the spongy, richly vascular, cancellous bone of the vertebral body. The end plates thus act to attach adjacent vertebrae to the disc. In other words, a transitional zone is created by the end plates between the malleable disc and the bony vertebrae.

The annulus of the disc is a tough, outer fibrous ring which binds together adjacent vertebrae. The fibrous portion, which is much like a laminated automobile tire, measures about 10 to 15 millimeters in height and about 15 to 20 millimeters in thickness. The fibers of the annulus consist of fifteen to twenty overlapping multiple plies, and are inserted into the superior and inferior vertebral bodies at roughly a 40-degree angle in both directions. This configuration particularly resists torsion, as about half of the angulated fibers will tighten when the vertebrae rotates in either direction, relative to each other. The laminated plies are less firmly attached to each other. The nucleus is immersed within the annulus, positioned somewhat like the liquid core of a golf ball. The healthy nucleus is largely a gel-like substance having high water content, and like air in a tire, serves to keep the annulus tight yet flexible. The nucleus-gel moves slightly within the annulus when force is exerted on the adjacent vertebrae while bending, lifting, etc.

The spinal disc may be displaced or damaged due to trauma or a disease process. A disc herniation occurs when the annulus fibers are weakened or torn and the inner tissue of the nucleus becomes permanently bulged, distended, or extruded out of its normal, internal annulus confines. The mass of a herniated or “slipped” nucleus tissue can compress a spinal nerve, resulting in leg pain, loss of muscle control, or even paralysis. Alternatively, with discal degeneration, the nucleus loses its water binding ability and deflates, as though the air had been let out of a tire. Subsequently, the height of the nucleus decreases causing the annulus to buckle in areas where the laminated plies are loosely bonded. As these overlapping, laminated plies of the annulus begin to buckle and separate, either circumferential or radial annular tears may occur, which may contribute to persistent and disabling back pain. Adjacent, ancillary spinal facet joints will also be forced into an overriding position, which may create additional back pain.

Whenever the nucleus tissue is herniated or removed by surgery, the disc space will narrow and may lose much of its normal stability. In many cases, to alleviate back pain from degenerated or herniated discs, the nucleus is removed and the two adjacent vertebrae are surgically fused together. While this treatment alleviates the pain, all discal motion is lost in the fused segment. Ultimately this procedure places a greater stress on the discs adjacent to the fused segment as they compensate for lack of motion, perhaps leading to premature degeneration of those adjacent discs.

As an alternative to vertebral fusion, a prosthetic spinal disc nucleus device can be implanted into the disc space, such as the HydraFlex™ nucleus replacement device available from Raymedica, LLC of Bloomington, Minn. With these and other spinal nucleus prostheses, the implantation procedure generally entails forming a passage through the annulus for insertion of the prosthesis. One surgical concern is the potential damage imparted upon the annulus during implantation surgery. The normal annular plies act to keep the annulus tight about the nucleus. During prosthetic nucleus implantation surgery, a surgical knife or tool is used to completely sever some portion of the annulus and/or remove an entire section or a “plug” of the annulus tissue. Adjacent vertebrae are often distracted, or spread apart, with a spinal implant fitted in the annular space. During distraction and implant insertion additional damage to the remaining annulus, as well as the vertebral endplates can occur. Additionally, when an entire section of the annulus is cut or removed to insert an implant, the layers making up the annulus often “flay” and/or “pull back” and the constraining or tightening ability of that portion of the annulus is lost. Similar concerns arise with numerous other prosthetic implantation procedures apart from the spinal disc, as well as with many bodily joint preparation procedures. More generally, then, surgeons have a need for surgical tools and methods that facilitate distraction of, and access to, an anatomically closed space (e.g., a joint) in a non-traumatic fashion.

### SUMMARY

Some aspects of the present disclosure relate to a surgical distractor for distracting a joint space so as to facilitate, for example, passage of a surgical tool into the joint space. With this in mind, the distractor includes a first arm, a second arm, a coupling device, and a limit device. Each of the arms includes a handle portion and a flange. The coupling device pivotably couples the first and second arms such that the flange of the first arm is adjacent the flange of the second arm. With this construction, the flanges combine to define a passage, with the distractor being configured to provide at least first and second states of expansion. In this regard, a size of the passage increases in transitioning of the distractor from the first state of expansion to the second state of expansion. Finally, the limit device is associated with the arms for selectively preventing transitioning of the distractor from the second state of expansion to the first state of expansion. In some embodiments, the flanges, and thus the passage, are laterally and/or angularly offset from the corresponding handle portions. In yet other embodiments, each of the arms further forms a contact face positioned proximally of, and extending transversely beyond, the corresponding flange.

Other aspects of the present disclosure relate to a method of surgically interfacing with a bodily joint. The method includes providing a surgical distractor including opposing arms each having a flange that combine to define a passage. The distractor device is transitionable between at least a first state of expansion and a second state of expansion, with a size of the passage being greater in the second state of expansion as compared to the first state of expansion. With this in mind, the distractor is arranged in the first state of expansion, and the flanges are introduced through an access site of the bodily joint. A distraction force is applied to the bodily joint by forcibly transitioning the distractor from the first state of expansion to the second state of expansion. Finally, the bodily joint is accessed via the passage. In some embodiments, the flanges combine to define a reverse wedge shape in transitioning to the second state of expansion, thereby drawing the flanges into the bodily joint. In yet other embodiments, the bodily joint is a spinal disc space, and accessing the bodily joint includes implanting a spinal nucleus prosthesis into the disc space via the passage.

### BRIEF DESCRIPTION OF THE DRAWINGS

FIG. 1 is a perspective view of a distractor, according to principles of the present disclosure.

FIG. 2 is an exploded, perspective view of a portion of the distractor of FIG. 1, according to principles of the present disclosure.

FIG. 3 is a side view of the distractor of FIG. 1, according to principles of the present disclosure.

FIG. 4 is a top view of a portion of the distractor of FIG. 1, according to principles of the present disclosure.

FIG. 5 is a cross-sectional view of a portion of the distractor of FIG. 1, according to principles of the present disclosure.

FIG. 6 is a side view of a portion of the distractor of FIG. 1, according to principles of the present disclosure.

FIG. 7 is a front view of the distractor of FIG. 1, according to principles of the present disclosure.

FIG. 8 is a back, perspective view of the distractor of FIG. 1, according to principles of the present disclosure.

FIG. 9 shows a distractor in a first state of expansion, according to principles of the present disclosure.

FIGS. 10-12 illustrate use of the distractor with an exemplary anatomical structure.

### DETAILED DESCRIPTION

In view of the above background, various distractor, anatomical access tools, implantation system, and implantation method objectives and advantages have been identified, with some embodiments of the present disclosure addressing distractors, anatomical access tools, implantation systems, and methods of prosthetic implantation that are characterized by one or more of the following: forming a passage through which surgical tool(s) (e.g., prosthesis, surgical instrument, etc.) can pass into an anatomically closed structure (e.g., a bodily joint such as a nucleus cavity); being substantially atraumatic to anatomical structural elements (e.g., endplates of a spinal disc space); for procedures entailing implantation of a prosthesis, reducing the force required to insert an implant into anatomically closed structure (e.g., bodily joint such as the nucleus cavity); substantially reducing or substantially preventing expansion of a joint access incision (e.g., an annulotomy); and/or substantially reducing a need for, or substantially obviating fixation on anatomy surrounding the bodily joint. In particular, it should be understood that various other objectives and advantages are also contemplated, and that the examples presented above are not exclusive.

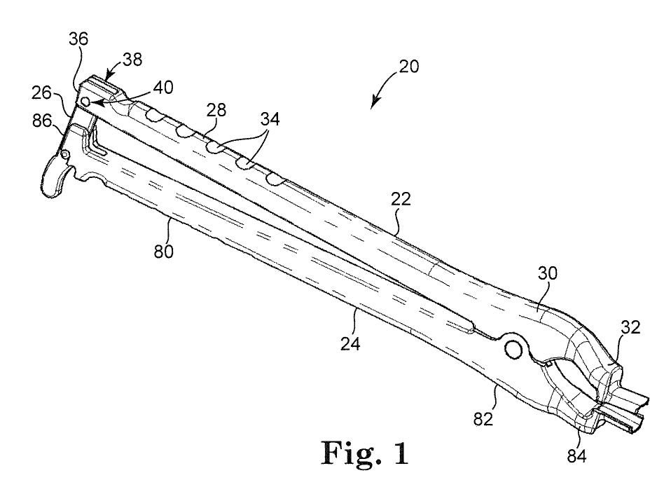

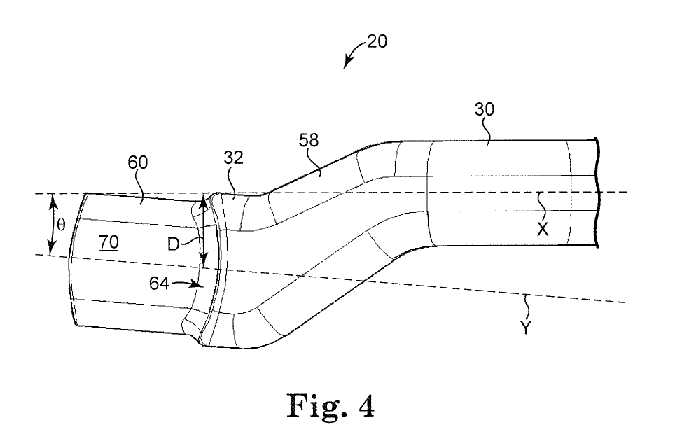

FIG. 1 is a perspective view of a distractor 20 according to some embodiments. In general terms, the distractor 20 is adapted for vertebral separation during surgical tool insertion, such as implantation of a nucleus prosthesis. Commensurate with the above explanation, the distractor 20 is not limited to prosthesis implantation applications, nor is it limited to the anatomy of the spinal disc. For reference, the terms “prosthetic” and “implant” are used interchangeably herein. The distractor 20 includes each of a first arm 22, a second arm 24, and a limit arm 26. The distractor 20 also defines a body axis X (FIG. 4) and an insertion axis Y (FIG. 4). The body axis X is defined centrally and longitudinally (in a lengthwise direction) between the first and second arms 22, 24 proximal to where the distractor 20 is grasped and manipulated by a user (not shown), while the insertion axis Y is defined centrally and longitudinally (in a lengthwise direction) between the first and second arms 22, 24 where the distractor 20 guides and/or facilitates surgical tool insertion, as will be understood in greater detail with reference to the text that follows.

The first arm 22 defines a first handle portion 28, a first hinge portion 30, and a first jaw portion 32. In some embodiments, the first arm 22 is optionally formed as a substantially monolithic, or unitary, piece. However, a plurality of separate, connected components, including separate, connected subcomponents associated with the first handle portion 28, the first hinge portion 30, and/or the first jaw portion 32, are used in other embodiments.

In some embodiments, the first handle portion 28 is substantially elongate and is generally adapted for grasping, for example including various ergonomic or other grasping/handling features. The first handle portion 28 optionally has indentations 34 formed over a portion thereof for ease and surety of grasping. For reference, a length of the first handle portion 28 can be selected according to a desired mechanical advantage, among additional or alternative design considerations. The first handle portion 28 defines a proximal end 36 of the first handle portion 28, with a slot 38 and shaft hole 40 formed toward the proximal end 36 of the first handle portion 28. The slot 38 is adapted to receive part of the limit arm 26 and the shaft hole 40 provides part of means for rotatably connecting the limit arm 26 to the first arm 22 as seated in the slot 38.

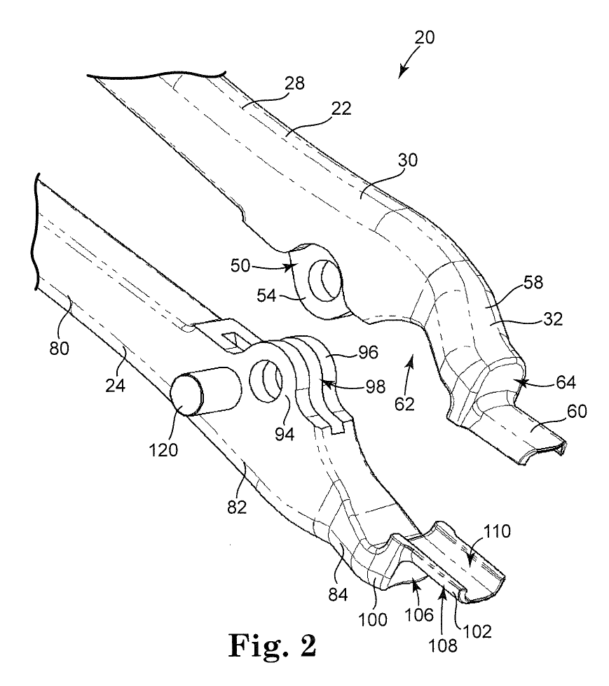

With reference to FIG. 2, the first hinge portion 30 extends contiguously between the first handle portion 28 and the first jaw portion 32, and is configured to provide part of a hinging means between the first arm 22 and the second arm 24. The first hinge portion 30 defines a first hinge well 50, a second hinge well (not shown), and a central hinge tab 54. The first hinge well 50 and second hinge well can be formed as semi-circular recesses on opposite sides of the central hinge tab 54, which, in turn, is formed as a substantially flat, semi-circular, and holed projection in some embodiments. As described below, the hinge tab 54 is sized and shaped in accordance with corresponding features of the second arm 24, establishing a pivoting or rotatable coupling between the first and second arms 22, 24. Alternatively, a wide variety of other configurations can be employed to establish the hinged coupling (e.g., linkages, etc.).

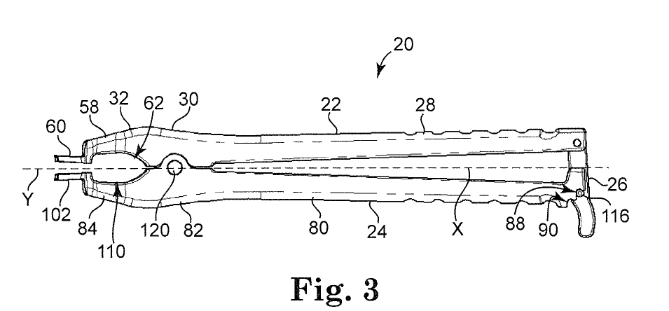

With additional reference to FIG. 3, in some embodiments, the first jaw portion 32 defines or forms a first transition 58 and a first flange 60. When viewed from a side profile, the first transition 58 slopes downwardly (relative to the orientation of FIG. 3) and defines a substantially u-shaped recess 62. Further, and with reference to FIG. 4, in some embodiments, the first transition 58 defines a contact face 64 of the jaw portion 32. The contact face 64 is substantially concave, corresponding generally to a convex shape of an expected anatomical structure encountered during use (e.g., a vertebrae and/or annulus), although other shapes are also contemplated. The first transition 58 is sized and shaped or otherwise adapted to result in a “jog,” or lateral offset D, and/or an angular offset θ of the first flange 60 in distal extension relative to the first handle portion 28 and the first hinge portion 30, which correspond to a lateral offset and an angular offset between the body axis X and the insertion axis Y in some embodiments. As shown in FIG. 4, in some embodiments, the first transition 58 is substantially S-shaped when viewed from the top in order to accomplish such lateral and/or angular offsets.

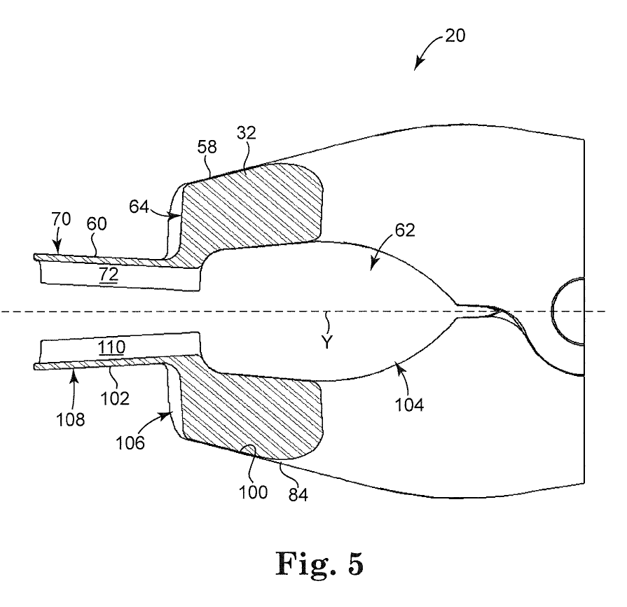

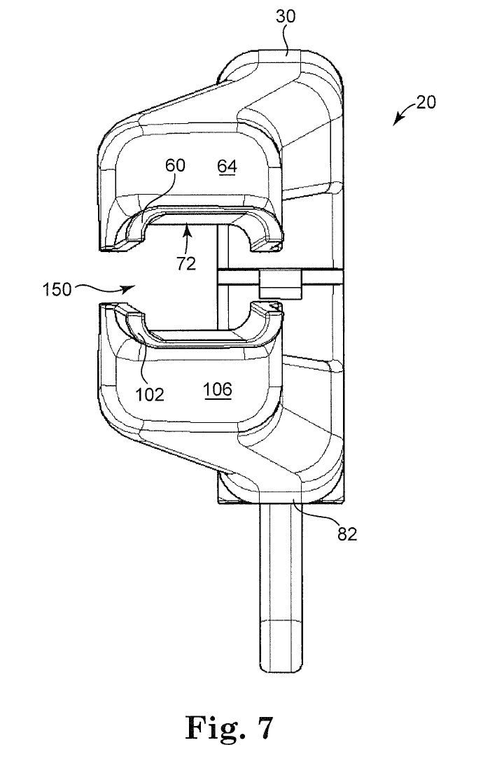

With additional reference to FIG. 5, in some embodiments, the first flange 60 is adapted to angle upwardly (relative to the orientation of FIG. 5), outwardly away from the insertion axis Y when the distractor 20 is operated (e.g., expanded) to accomplish joint distraction, as explained in greater detail below. In some embodiments, the first flange 60 is u-shaped in longitudinal extension (i.e., transverse cross-section), having an outer surface 70 (see also, FIG. 4) and a curved inner surface 72 (see also, FIG. 7) defining a portion of a passage adapted to slidably contact a surgical tool such as a spinal prosthetic. For example, the inner surface 72 is substantially smooth and/or otherwise exhibits a reduced friction characteristic, as described in greater detail below. However, it is also contemplated that in some embodiments, the inner surface 72 is adapted to grip or otherwise have a relatively high friction characteristic. Regardless, by employing a u-shape with some embodiments, bending resistance of the first flange 60 is increased while thickness can be minimized according to a desired moment of inertia, among other additional or alternative factors. For example, in some embodiments, the first flange 60 is about 0.75 mm thick. By reducing the thickness of the first flange 60 (as compared to conventional joint access tools), and corresponding portions of the second arm 24, the distractor 20 defines a smaller profile, which allows less intrusive distraction and/or implant insertion through a surgical incision (e.g., an annulotomy), as will be described in greater detail.

Returning to FIG. 1 and turning to the second arm 24, in some embodiments, the second arm 24 is substantially similar to the first arm 22, with portions of the second arm 24 being a substantial mirror image of the first arm 22 and portions of the second arm 24 being adapted to mate with the first arm 22 in a hinging manner. For example, the second arm 24 defines a second handle portion 80, a second hinge portion 82, and a second jaw portion 84. As with the first arm 22, the second arm 24 is optionally a single piece or alternatively multiple connected components.

In some embodiments, the second handle portion 80 is also substantially elongate and is generally adapted for grasping, for example including various ergonomic or other grasping/handling features similar, or different from the first handle portion 28 as desired. A length of the second handle portion 80 can also be selected according to a desired mechanical advantage, among additional or alternative design considerations. The second handle portion 80 defines a proximal end 86.

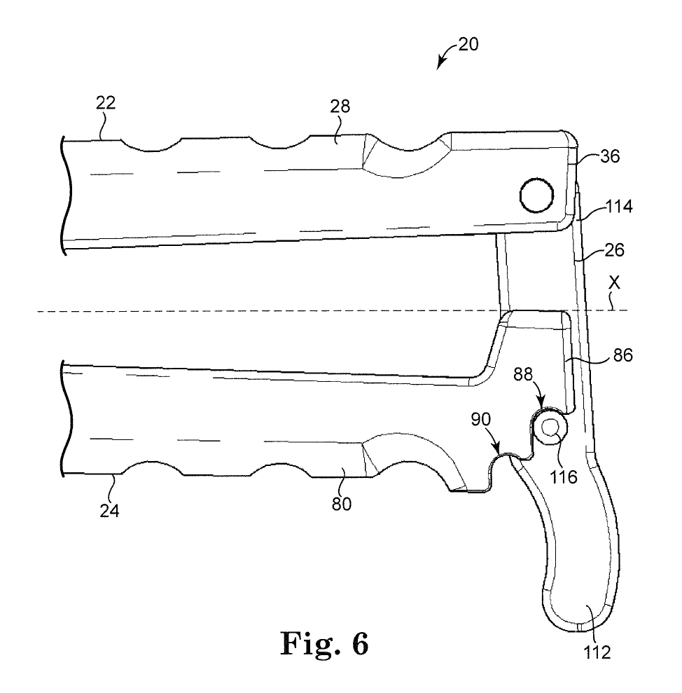

With reference to FIG. 6, in some embodiments, the second handle portion 80 defines a first limit seat 88 at a first offset from the body axis X and a second limit seat 90 at a second, greater offset from the body axis X. As will be described in greater detail, the first and second limit seats 88, 90, respectively, are optionally used with the limit arm 26 to set the distractor 20 at a desired distraction distance or otherwise resist collapsing of the distractor 20 during a joint distraction procedure.

With reference to FIG. 2, in some embodiments, the second hinge portion 82 defines a first outer hinge tab 94, a second outer hinge tab 96, and a central hinge well 98. The first and second outer hinge tabs 94 are opposingly positioned relative to the central hinge well 98, spaced to receive the central hinge tab 54 of the first hinge portion 30, and each being formed as a substantially flat, semi-circular, and holed projection. In turn, the central hinge well 98 is formed to receive the central hinge tab 54 such that the first and second outer hinge tabs 94, 96 substantially “line up” with the central hinge tab 54 upon assembly, as will be described in greater detail. Alternatively, the second hinge portion 82 can assume a wide variety of other forms (and/or include additional components and/or mechanisms) capable of establishing a pivotable coupling with corresponding feature(s) of the first arm 22.

With reference between FIGS. 2 and 5, in some embodiments, the second jaw portion 84 is similar to the first jaw portion 32, being a substantially complementary, mirror image thereof. Thus, the second jaw portion 84 optionally has complementary features to the first jaw portion 32, including, for example, a complementary lateral offset, or jog, and/or an angular offset such as those previously described in association with the first transition 58 and the first flange 60. With this in mind, in some embodiments, the second jaw portion 84 defines a second transition 100 and a second flange 102, where the second transition 100 defines a recess 104 and a contact face 106, and the second flange 102 defines an outer surface 108 and an inner surface 110, each of which is substantially complementary, or a mirror image of, corresponding features of the first jaw portion 32. As alluded to above, the second flange 102 is optionally relatively thin-walled in view of forces associated with distraction, for example with the second flange 102 having a thickness of about 0.75 mm.

With reference to FIG. 6, the limit arm 26 defines a first end 112 and a second end 114 and forms or includes a laterally projecting stop 116 configured to be seated in the first and second limit seats 88, 90. The first end 112 is optionally formed in a “trigger” shape to facilitate ease of grasping and use. In turn, the second end 114 is adapted to be hingedly secured at the proximal end 36 and in the slot 38 (FIG. 1) of the first handle portion 28. The stop 116 is optionally a cylindrical projection, boss, or other feature suited for interacting with the first and second limit seats 88, 90 to secure the distractor 20 at a desired distraction distance, or otherwise assist in preventing the first and second handle portions 28, 80 from moving away from one another when engaged.

With reference between FIGS. 2 and 3, and in view of the above, assembly of the distractor 20 in some embodiments includes rotatably hinging the first and second hinge portions 30, 82 together by mating the first and second outer hinge tabs 94, 96 with the inner hinge tab 54 and securing a pin 120 through each of the respective hinge tabs 54, 94, 96. In this manner, the pin 120 optionally acts as a fulcrum point between the first and second handle portions 28, 80 and the first and second jaw portions 32, 84, where squeezing the handle portions 28, 80 results in a relative widening at the jaw portions 32, 84. The hinge location (i.e., point of coupling between the hinge portions 30, 82 via the pin 120) relative to the flanges 60, 102 is selected to ensure a level of non-parallel alignment of the flanges 60, 102 at a final or maximum distraction (or expansion) position relative to the bodily joint to which the distractor 20 is applied. For example, in some embodiments, the distractor 20 is configured to exert and maintain a distraction force of at least 1000 Newtons (N) at the first and second flanges 60, 102 with a grip force (i.e., a force that must be exerted at the handle portions 28, 80 where a person would grasp) requirement of about 300N or less, although other distraction forces and/or grip forces are also contemplated. Additionally, the limit arm 26 is rotatably secured in the slot 38 (FIG. 1) of the first handle portion 28 in such a manner that that the stop 116 can be releasably received in ether of the first or second limit seats 88, 90.

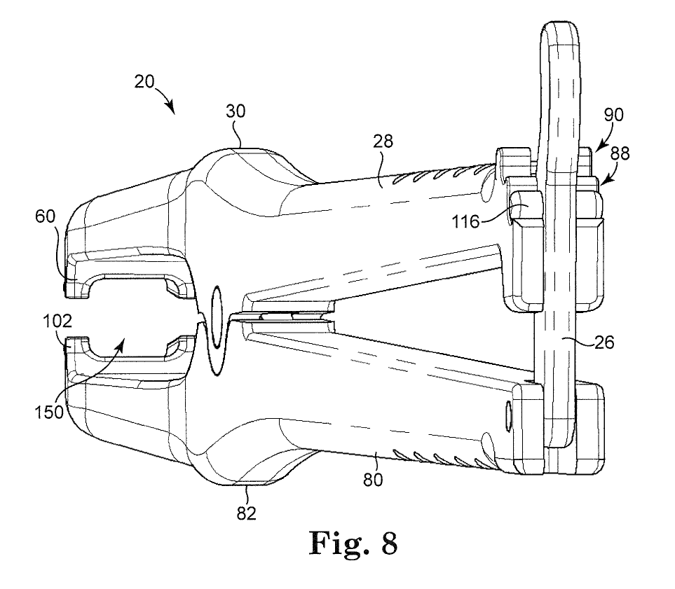

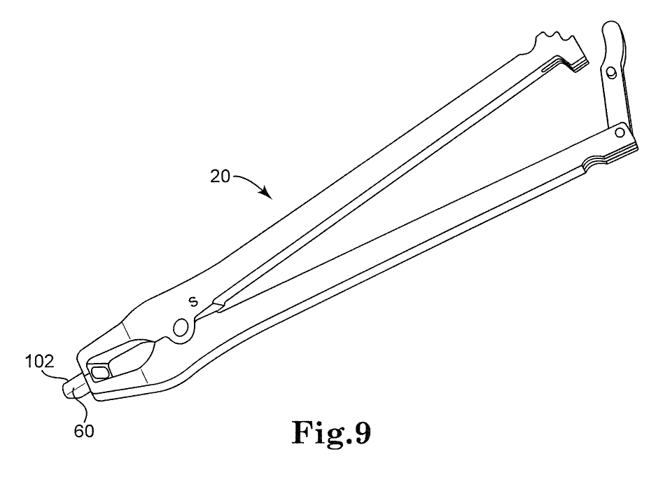

With reference between FIGS. 7 and 8, in some embodiments, the first and second flanges 60, 102 combine to define a passage 150. A size (e.g., height) of the passage 150 changes as the flanges 60, 102 move toward and away from one another, and in particular where the distractor 20 is opened and closed using the handle portions 28, 80. Thus, the distractor 20 defines various states of expansion at the first and second flanges 60, 102. In some embodiments, the passage 150 is minimized in size when the distractor 20 is in a first state of expansion, with each of the flanges 60, 102 contacting (FIG. 9) upon pulling the handle portions 28, 80 away from one another to a greatest allowable extent.

In some embodiments, the distractor 20 is configured such that in the first state of expansion, a maximum size (e.g., height) of the passage 150 is commensurate with or smaller than that of a surgical tool (not shown) to be used with the distractor 20 in performing a particular procedure (e.g., a prosthesis). FIG. 9 illustrates the distractor 20 with the flanges 60, 102 fully collapsed in the first state of expansion. Notably, in the first state of expansion, an overall outer profile collectively defined by the flanges 60, 102 is minimized according to some embodiments.

With reference back to FIGS. 7 and 8, generally, upon squeezing the handle portions 28, 80 (FIG. 8), the distractor 20 is transitioned to a second state of expansion in which a maximum opening size of the passage 150 is greater than that in the first state of expansion. For example, the distractor 20 is configured such that in the second state of expansion, the passage 150 is sized and shaped to allow reception and/or full passage of a surgical tool (not shown) through the passage 150. By way of reference, with prosthetic spinal disc nucleus implantation procedures, the passage 150 is sized to permit longitudinal (e.g., sliding) movement of the prosthesis therethrough in the second state of expansion.

The distractor 20 is also configured to provide a third state of expansion in which a maximum opening size of the passage 150 is greater than that of the second state of expansion. As referenced above, in some embodiments, the first and second flanges 60, 102 are angled to “flare away” from one another in at least the second and/or third states of expansion via shapes of the flanges 60, 120 and location of the hinge point (i.e., the pin 120). If desired, the distractor 20 can be configured such that the first and second flanges 60, 102 optionally extend substantially parallel to one another, or even toward one another, in the first state of expansion to facilitate insertion of the first and second flanges 60, 102 into a surgical incision, with distraction of the joint in question occurring upon transition to the second and/or third states of expansion, as described in greater detail below. Alternatively, the distractor 20 can be configured such that the first and second flanges 60, 102 extend away from one another in the first state of expansion.

With additional reference to FIG. 8, the limit arm 26 is optionally used to lock or limit the distractor 20 in one or more states of expansion. For example, the limit arm 26 optionally releasably locks or limits the distractor 20 in the second state of expansion with the stop 116 in the first limit seat 88 as shown in FIG. 8, and releasably locks or limits the distractor 20 in the third state of expansion with the stop 116 in the second limit seat 90, which is not actually shown in FIG. 8, but can be understood with reference thereto.

Additionally, as can be seen in FIGS. 7 and 8, in some embodiments, the passage 150 is angularly and laterally offset relative to the handle and/or hinge portions 28, 30, 80, 82, or in alternative terms, the passage 150 is laterally offset and angled relative to the body axis X (FIG. 4). In this manner, the passage 150 is not longitudinally aligned with a remainder of the distractor 20, such that users of the distractor 20 readily view, and gain access to, the passage 150 at an offset “angle of attack.” This can help reduce viewing and handling interference from hands of the user (not shown), instruments, or other objects, for example, during joint distraction and/or delivery of surgical tool via the passage 150.

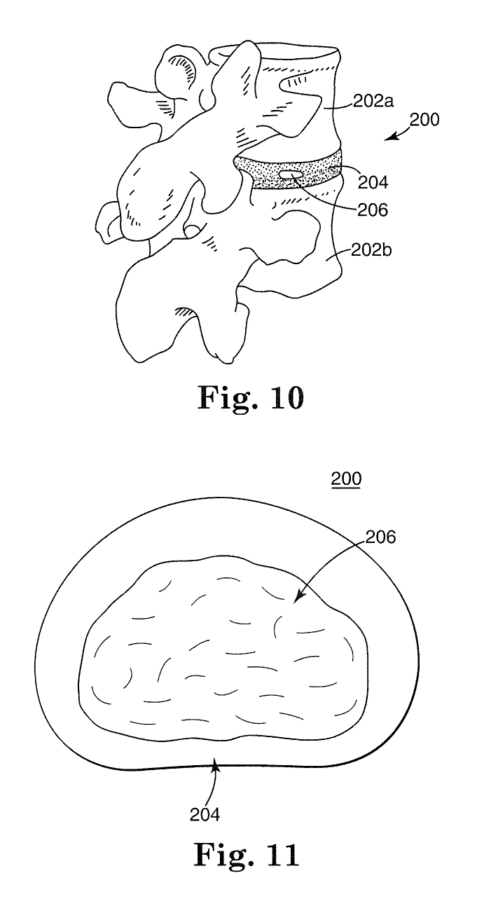

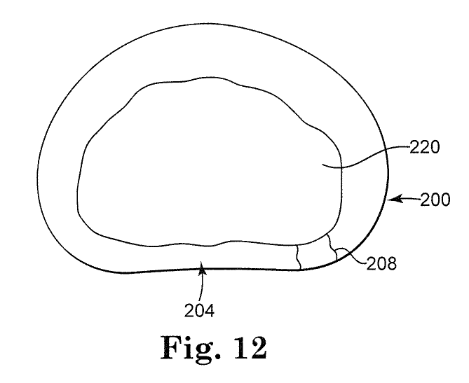

The distractor 20 can be used in performing a wide variety of surgical procedures in which access to and/or distraction of an anatomically closed structure, such as a bodily joint, is required. One non-limiting example is in connection with implantation of a nucleus prosthesis into a spinal disc space. One such procedure is described in detail in U.S. Provisional Application Ser. No. 60/846,944, filed Sep. 25, 2006, the teachings of which are incorporated herein by reference. In general terms, and with reference to FIGS. 10 and 11, a spinal disc space 200 is defined between opposing vertebrae 202a, 202b, and includes an annulus 204 and opposing endplates (hidden in FIGS. 10 and 11, but defined by the opposing vertebrae 202a, 202b) surrounding a nucleus 206. As shown in FIG. 12, an incision or hole 208 (e.g., a partial annlutomy) is formed in the annulus 204, and the nucleus 206 (FIG. 10) is partially or completely removed.

With additional reference to FIG. 3, the distractor 20 is then delivered to the disc space 200. In particular, the distractor 20 is transitioned to the first state of expansion, such that the flanges 60, 102 are in close proximity to one another, defining a minimized profile. In the first state of expansion, the flanges 60, 102 are readily inserted into the hole 208. The contact faces 64, 106 (FIG. 7) contact the annulus 204, serving as a stop to further distal movement and positioning the flanges 60, 102 at desired locations for subsequent interaction with the disc space 200. The distractor 20 is then transitioned to the second (or third) state of expansion, causing the flanges 60, 102 to separate from one another. Due to the distally flared shape of the flanges 60, 102, the flanges 60, 102 contact the opposing end plates (one of which is shown generally at 220 in FIG. 12) via positioning dictated by the interface between the annulus 204 and the contact faces 64, 106, and exert a distraction force thereon. The distraction forces are thus focused upon the endplates 220, with minimal distraction occurring at the access site/hole 208 in the annulus 204. Notably, the shape of the flanges 60, 102 in combination with the hinge point (e.g., the pin 120) of the distractor 20 effectuates a reverse wedge interface between the flanges 60, 102 and the annulus 204 in transitioning to the second (or third) state of expansion, thus drawing the flanges 60, 102 into the disc space 200 (as opposed to forcing or ejecting the flanges 60, 102 from the disc space 200). The so-generated distraction force causes the vertebrae 202a, 202b to separate from one another. The limit arm 26 is engaged to lock the distractor 20 in the second (or third) expansion state.

In the second (or third) state of expansion, a prosthetic spinal disc nucleus (not shown) is then delivered through the passage 150 and distally into the disc space 200. In this regard, one or more additional surgical tools or instruments can be delivered through the passage 150 to assist in desired placement of the prosthesis. The off-set longitudinal positioning of the flanges 60, 102 relative to the corresponding handle portions 28, 80 locates the handle portions 28, 80 away from the surgical site (e.g., the hole 208) such that the surgeon's view of the surgical site is only minimally obstructed. Where desired or necessary, the distractor device 20 can be further transitioned from the second expansion state to the third expansion state to effectuate enhanced distraction of the vertebrae 202a, 202b. Upon completion of the procedure, the limit arm 26 is disengaged, allowing the distractor 20 to revert to the first expansion state. The distractor 20 can then be removed from the disc space 200.

As referenced above, in some embodiments use of the distractor 20 provides a smooth sliding surface (e.g., the inner surfaces 72, 110 shown in FIG. 5) for conveying the surgical tool(s) into the anatomically closed structure (e.g., the disc space 200). By forming the passage 150 through which the surgical tool(s) can pass, a distraction and/or implantation method that is substantially atraumatic to anatomical structures (e.g., the annulus 204 and the endplates 220) and entails a reduced insertion force is provided in some embodiments. Additionally, a single access site (e.g., the hole 208) can be used both for insertion of the surgical tool(s) into the anatomical structure, as well as distraction if desired. However it should also be understood that multiple surgical tools (e.g., multiple implants) are inserted and/or multiple access sites are formed in some embodiments. Regardless, in some embodiments the minimized profile of the flanges 60, 102 in the first state of expansion serves to substantially reduce or prevent inelastic expansion of the access site. Additionally, the minimized flange profiles allow use of a relatively smaller size access site, aiding in healing and/or reducing other unwanted effects on the anatomical structure through which the access site is formed. Flange design also helps reduce likelihood of the distractor 20 ejecting from the site during distraction and/or implant insertion. Additionally, in some embodiments, the design of the distractor 20, which allows insertion of the distractor 20 into the access site to accomplish distraction, helps eliminate or reduce a need for fixation of instruments on anatomy surrounding the access site to accomplish effective distraction.

Although the present disclosure has been described with reference to preferred embodiments, workers skilled in the art will recognize that changes can be made in form and detail without departing from the spirit and scope of the present disclosure.

What is claimed is:

1. A surgical distractor for distracting a joint space to facilitate passage of a surgical tool into the joint space, the distractor comprising:
a first arm including a handle portion and a flange;
a second arm including a handle portion and a flange;
a coupling device pivotably coupling the first and second arms, with the flange of the first arm being adjacent the flange of the second arm;
wherein the flanges combine to define a passage and the surgical distractor is configured to provide a first state of expansion and a second state of expansion, a size of the passage being greater in the second state of expansion than in the first state of expansion; and
a limit device associated with the arms for selectively preventing transitioning of the distractor from the second state of expansion to the first state of expansion.

2. The distractor of claim 1, wherein each of the flanges are laterally offset from the corresponding handle portions.

3. The distractor of claim 2, wherein the handle portions combine to define a first longitudinal axis and the flanges combine to define a second longitudinal axis, and further wherein the first longitudinal axis is laterally offset from the second longitudinal axis.

4. The distractor of claim 3, wherein the first and second longitudinal axes are parallel.

5. The distractor of claim 3, wherein the first and second longitudinal axes are non-parallel such that the passage is angularly offset relative to the handle portions.

6. The distractor of claim 1, wherein a pivot point of the first and second arms is formed intermediate the respective flanges and handle portions such that each of the flanges extend distally relative to the pivot point from a proximal side to a distal side, and further wherein each of the flanges project transversely outwardly in distal extension from the proximal side to the distal side in at least the second state of expansion.

7. The distractor of claim 1, wherein each of the flanges are U-shaped in transverse cross-section.

8. The distractor of claim 1, wherein at least a portion of the passage is arcuate in transverse cross-section.

9. The distractor of claim 1, wherein each of the arms further includes a transition disposed between the corresponding flange and the handle portion, the transition defining a contact face extending transversely beyond the corresponding flange.

10. The distractor of claim 9, wherein the transition establishes a transverse offset of the flange relative to the corresponding handle portion.

11. The distractor of claim 1, wherein the distractor is configured such that the flanges combine to define a reverse wedge shape in at least the second state of expansion.

12. The distractor of claim 1, wherein the limit device includes a limit arm pivotably coupled to the first arm.

13. The distractor of claim 12, wherein the limit device further includes a pin extending from the limit arm, and further wherein the handle portion of the second arm forms a seat sized to selectively receive the pin.

14. A method of surgically interfacing with a bodily joint, the method comprising:
providing a surgical distractor including:
a first arm including a handle portion and a flange,
a second arm including a handle portion and a flange,
a coupling device pivotably coupling the first and second arms, with the flange of the first arm being adjacent the flange of the second arm,
wherein the flanges combine to define a passage and the surgical distractor is configured to provide a first state of expansion and a second state of expansion, a size of the passage being greater in the second state of expansion than in the first state of expansion,
a limit device associated with the arms for selectively preventing transitioning of the distractor from the second state of expansion to the first state of expansion;
arranging the distractor in the first state of expansion;
introducing the flanges through an access site of the bodily joint;
applying a distraction force to the bodily joint by forcibly transitioning the distractor to the second state of expansion; and
accessing the bodily joint via the passage.

15. The method of claim 14, wherein applying a distraction force includes arranging the flanges to define a reverse wedge shape to draw the flanges into the bodily joint.

16. The method of claim 14, wherein each of the arms further includes a contact face proximal the corresponding flange, and further wherein introducing the flanges includes abutting the contact faces against an anatomical structure of the bodily joint.

17. The method of claim 14, wherein the flanges are laterally offset from the corresponding handle portions, and further wherein accessing the bodily joint includes inserting a surgical tool through the passage, with a proximal portion of the surgical tool being adjacent the handle portions.

18. The method of claim 14, wherein the bodily joint is a spinal disc space.

19. The method of claim 18, wherein accessing the bodily joint includes implanting a spinal nucleus prosthesis into the disc space via the passage.

20. The method of claim 18, wherein applying a distraction force is characterized by the absence of a fixed connection between the distractor and anatomy outside of the disc space.
